(19) United States
(12) Patent Application Publication
(10) Pub. No.: US 2015/0328364 A1
(43) Pub. Date: Nov. 19, 2015

(54) A COMPOSITION FOR MAKING A CEMENT OR AN IMPLANT

(71) Applicant: QUEEN MARY AND WESTFIELD COLLEGE, London (GB)

(72) Inventors: Robert Graham Hill, Berkshire (GB); Natalia Karpukhina, Greater London (GB); Niall Kent, Bedfordshire (GB)

(73) Assignee: QUEEN MARY AND WESTFIELD COLLEGE, London (GB)

(21) Appl. No.: 14/367,379

(22) PCT Filed: Dec. 21, 2012
(86) PCT No.: PCT/EP2012/076844

§ 371 Date: Jun. 20, 2014

(30) Foreign Application Priority Data
Dec. 23, 2011 (GB) 1122257.7

(51) Int. Cl. A61L 27/10; A61L 27/54; A61L 27/58; A61L 27/12

(52) U.S. Cl. CPC A61L 27/10; A61L 27/12; A61L 27/54; A61L 27/58; A61L 2430/02

(57) ABSTRACT

A composition for making a cement or an implant, the composition comprising a silicate glass and at least one compound selected from the group consisting of a calcium phosphate salt, a strontium phosphate salt and a phosphate glass.

The present invention relates to a composition for making a cement or an implant.

Cements are made from compositions which, when mixed with water, form a workable paste. This paste can be moulded to fill the contours of an implantation site such as a tooth socket or a bone defect, and then sets in situ in the implantation site. An implant or bone substitute may also be made from a cement but the composition from which the implant is made is moulded into shape and allowed to set before being inserted into the implant site within the body.

Calcium phosphate cements (CPCs) were invented by Chow and Brown and are now widely used in various forms.

The first CPC developed by Chow and Brown consisted of a composition comprising equimolar amounts of ground Ca₄(PO₄)₂O (tetracalcium phosphate, TTCP) and CaHPO₄ (dicalcium phosphate anhydrous, DCPA). When mixed with water, this composition forms a workable paste, which can be moulded during surgery to fit the contours of the implantation site. This cement hardens within 30 minutes forming nanocrystalline hydroxyapatite (HA) as the product. The reaction is isothermic and occurs at physiological pH so tissue necrosis does not occur during the setting reaction.

HA is the primary inorganic component of natural bone and tooth, and so is biocompatible and osteoconductive. Over time, the hardened CPC is gradually remodeled, resorbed and replaced with new bone tissue. The first CPC was approved for the treatment of non-load-bearing bone defects in 1996.

CPCs have two significant advantages over pre-formed, sintered ceramics: 1.) the CPC paste can be shaped during surgery to fit the contours of the implantation site. 2.) the nanocrystalline hydroxyapatite structure of the CPC makes it osteoconductive causing it to be gradually resorbed and replaced with new bone.

Accordingly, since the original invention of the first CPC by Chow and Brown, numerous variants of that CPC have been developed by combining different water-soluble calcium phosphate salts and then mixing these salts with water. For example, CPCs can be formed from mixtures of tricalcium phosphate and calcium carbonate, or DCPA and calcium hydroxide (TTCP-Ca₄(PO₄)₂) (73% mass fraction) and dicalcium phosphate. Generally, the calcium:phosphate ratio of these CPCs is chosen to be close to 1.67 because this is the stoichiometry of apatite and so using this ratio favours apatite formation.

Although CPCs are widely used, they do suffer from many problems. Thus, they exhibit sluggish setting characteristics, are susceptible to early ingress of body fluids, and have poor compressive strengths and flexural strengths. This means that the clinical applications of CPCs are restricted to non-load-bearing applications, such as dental and cranio-facial applications. The potential applications of CPCs would be markedly extended if their strength could be improved.

Another disadvantage of CPCs is that, because they are prepared using crystalline calcium phosphate salts, the cements are restricted to the stoichiometry ratios of those salts. It is for this reason that TTCP is frequently used in CPCs. TTCP has a Ca/P ratio of 2 and so is one of the few salts that has a Ca/P ratio above 1.67. If TTCP is combined with a calcium phosphate salt which has a Ca/P ratio below 1.67, then it is possible to obtain a composition having a Ca/P ratio close to 1.67.

A further disadvantage of CPCs is that soluble fluoride salts have a deleterious influence on cement properties. Incorporating fluoride into CPCs is desirable as it means that the cement is fluorapatite-based (FAP-based) rather than hydroxyapatite-based (HA-based). FAP is more resistant to acid dissolution in oral fluids than HA and aids in the prevention of dental caries. Moreover, fluoride irons are known to aid apatite formation and stimulate the cell division of osteoblasts, the bone forming cells.

Recent developments of CPCs have focused on improving mechanical properties by making the cement macroporous and seeding cells and growth factors into the cement.

There is another cement system besides apatite based CPCs. In this system, the cement sets to form DCPD, known as brushite. In 1987 Mirtchi and Lemaitre reported the formation of brushite cement from the reaction of beta tricalcium phosphate (β-TCP) and monocalcium phosphate monohydrate (MCPM). Bohner et al. produced a similar cement substituting MCPM with phosphoric acid. However, despite many reports of brushite cements in the literature, most published work has concentrated on optimising apatite-forming CPCs largely because, although their mechanical strength is poor, it is superior to that of other CPC cements.

It is an object of the invention to seek to mitigate the problems which have been found with existing cements.

Accordingly, the invention provides a composition for making a cement or an implant, the composition comprising a silicate glass and at least one compound selected from the group consisting of a calcium phosphate salt, a strontium phosphate salt and a phosphate glass.

The present invention utilizes mixtures of silicate glasses with calcium phosphate salts, strontium phosphate salts or phosphate glasses. Combining a silicate glass with a calcium phosphate salt, strontium phosphate salt or phosphate glass results in a composition which, when mixed with water, forms a workable paste, which can be moulded to fill the contours of an implantation site, and then sets in situ. Alternatively, this workable paste may be allowed to set before filling takes place, the set cement being ground into granular form, and then used to fill an implantation site. Another possibility is that the workable paste may be used to fill a mould, and then allowed to set in order to produce a pre-set implant of the required shape.

Preferably, the composition is such that, when mixed with water, it sets to form a hardened cement in under one hour.

The term “glass” as used in the claims is intended to incorporate both glasses and glass-ceramics. Glasses are entirely amorphous (i.e non-crystalline), whereas glass-ceramics have an amorphous phase and one or more crystalline phases.

Amorphous glasses and amorphous/crystalline glass ceramics have a number of advantages over the crystalline calcium phosphate salts used in CPCs.
1. Unlike a crystalline salt, the composition of an amorphous glass or amorphous/crystalline glass-ceramic is not limited by stoichiometry and can be varied at will.
2. Amorphous glasses and amorphous/crystalline glass ceramics are generally more reactive and dissolve more quickly than their crystalline counterparts since an amorphous phase is always in a higher energy state than its equivalent crystalline counterpart.
3. It is possible to incorporate many species into a glass or glass-ceramic for subsequent release that either can be incorporated or could not be released at a sufficient rate from calcium salts. Notable examples include strontium, zinc, cobalt and fluoride.
Addition of strontium is beneficial because strontium has been shown to inhibit bone resorption and promote bone formation by inhibiting osteoclasts and promoting osteoblasts making it desirable in conditions where bone is weak i.e osteoporosis. Strontium will also add a degree of radiopacity to the cements, which is a favourable property allowing the implanted cement to be observed radiographically by X-rays and enables the surgeon to follow the resorption of the cement or implant.
Zinc addition has two potential benefits. Firstly, it has been shown that, in small quantities, zinc significantly increases proliferation of human osteoblastic cells. Secondly, it is thought that zinc could promote healing because zinc is a cofactor in many enzymes in the body which affect healing times.
Cobalt has been shown to be able to induce angiogenesis and so its addition could be useful for certain applications.
Fluoride addition is beneficial because it should allow the formation of fluoroapatite (FAP). FAP is more resistant to acid dissolution in oral fluids than HA and aids in the prevention of dental caries. Moreover, fluoride ions are known to aid apatite formation and stimulate the cell division of osteoblasts, the bone forming cells. Fluorapatite cements are preferred for restorative dental fillings and for bone cements and substitutes where resorbtion by osteoclasts and remodeling is considered undesirable. In contrast octacalcium phosphate based cements and apatite cements are preferred where resorbtion of the cement in the body is preferred.

Preferably, the glass is degradable and/or bioactive.

Bioactive glasses were developed by Hench in the late 1960s. A bioactive glass is a silicate-based glass that dissolves in physiological fluids forming an apatite on its surface. Like CPCs, bioactive glasses are used as a bone substitute. They are much more resorbable than apatite-based CPCs and are also considered to stimulate new bone formation much more readily than CPCs. However, they are currently used clinically as granules or as a putty. In granular form, they lack the desirable mouldability and ability to set in situ of CPCs. In putty form, they have no inherent strength. By combining a bioactive glass with a calcium phosphate salt, a strontium phosphate salt or a phosphate glass, it becomes possible to obtain a cement having the mouldability and ability to set in situ of CPCs.

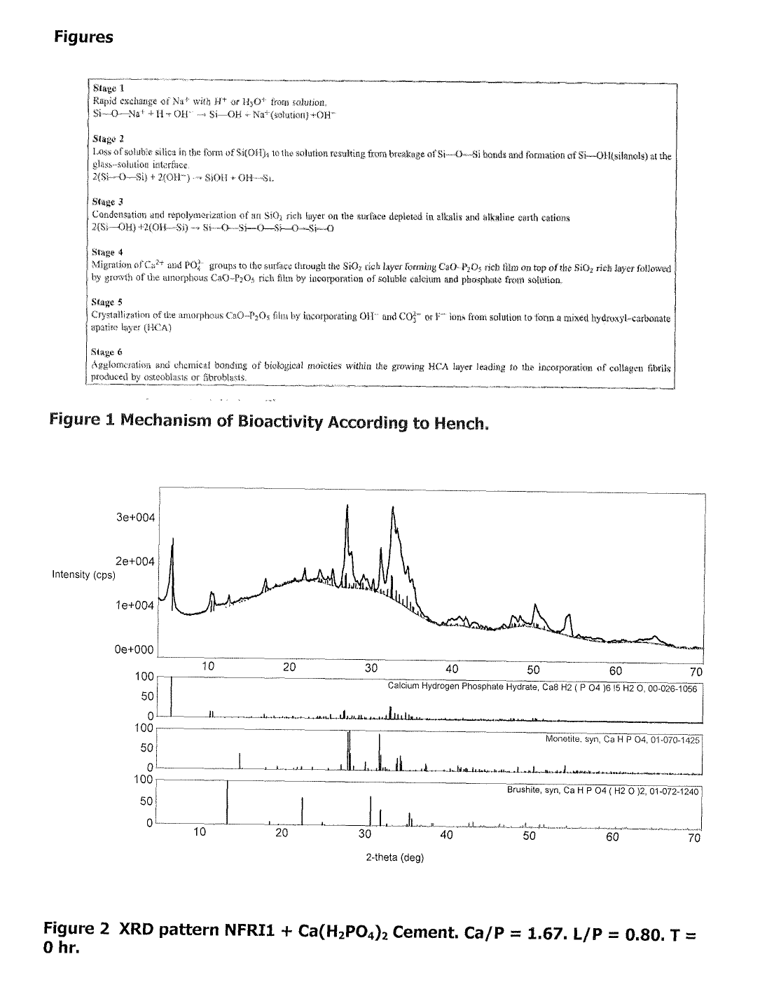

FIG. 1 shows the widely accepted mechanism proposed by Hench to explain the bioactivity of bioactive glasses. The first step is the ion exchange of Na⁺ ions from the glass for protons in the surrounding solution. From this mechanism, it would be expected that glasses with little or no sodium would not be bioactive or would exhibit limited bioactivity. There therefore appears to be limited possibility for forming cements according to the invention from sodium-free or low sodium silicate glasses. Surprisingly, however, the applicant has recently discovered new sodium-free or low sodium-containing silicate glasses that are exceedingly bioactive and form apatite in under 4 hours. Such glasses offer the possibility of forming a new generation of cements. The bioactivity (defined as the time to form an apatite like phase) of such sodium-free or low sodium glasses may be enhanced by adding fluoride to the glass and/or by increasing the phosphate content of the glass.

Accordingly, the silicate glass may contain less than 30 mole percent, preferably less than 20 mole percent, more preferably less than 10 mole percent, of an alkali metal oxide or less than 25 mole percent, preferably less than 12 mole percent, of an alkali metal fluoride.

Small amounts of alkali metals are desirable, however, as they reduce the melting temperature of the glass and facilitate melting. Accordingly, the silicate glass preferably contains an alkali metal oxide or an alkali metal fluoride.

The silicate glass may have a SiO₂ content below 60 mole percent.

The silicate glass may have a SiO₂ content between 20 and 55 mole percent, preferably between 35 and 50 mole percent.

The silicate glass may contain a fluoride, the fluoride content expressed as a divalent or monovalent fluoride being up to 25 mole percent, preferably up to 18 mole percent, more preferably between 0.01 and 12 mole percent, most preferably between 0.01 and 5 mole percent.

The silicate glass may contain a metal fluoride.

The silicate glass may contain 10 to 60 mole percent, preferably 20 to 55 mole percent, more preferably 35 to 50 mole percent of CaO or SrO or a combination thereof.

The (Ca+Sr):P/molar ratio of the silicate glass may lie between 0.1 and 20, preferably between 0.5 and 3.

The silicate glass is preferably produced by a high temperature melt quench route on the basis of cost and convenience but can be made via a sol gel route.

The composition may comprise a source of soluble phosphate. Alternatively, the phosphate may be included in the water which is then mixed with the composition to form the cement or implant.

The phosphate glass may have a P₂O₅ content between 25 and 60 mole percent.

The (Ca+Sr):P/molar ratio of the phosphate glass may be between 0.1 and 1.5.

The (Ca+Sr):P molar ratio of the phosphate glass may be between 0.4 and 0.8.

The glasses may be ground to a powder with a particle size less than 1 mm.

The glasses preferably have a particle size less than 100 microns, preferably less than 60 microns, more preferably less than 38 microns.

The composition may comprise a phosphate glass, and the phosphate glass and silicate glass may be co-sintered together to form a glass alloy at a temperature in the range 350-900° C.

The silicate glass and/or, where present, the phosphate glass may contain cobalt with a molar percentage from 0.01 to 4.0.

The phosphate glass may contain 10 to 60 mole percent, preferably 2 to 30 mole percent, more preferably 4 to 20 mole percent, strontium.

The calcium phosphate salt may be calcium hydrogen phosphate (Ca(H₂PO₄)₂ and/or CaHPO₄).

The strontium phosphate salt may be strontium hydrogen phosphate (Sr(H₂PO₄)₂ and/or SrHPO₄).

The phosphate salt may be a combination of strontium hydrogen phosphate and calcium hydrogen phosphate.

The phosphate glass and/or the silicate glass may together contain up to 5 mole percent zinc oxide or 10 mole percent zinc fluoride.

The composition may comprise an apatite such as hydroxyapatite or fluorapatite

The apatite may be crystallised to seed the nucleation and promote the crystallisation of an apatite like phase. The size of the crystals may be in the range 30 nm to 5 microns, preferably 30 nm to 3 microns.

As an alternative to adding seed apatite crystals to the composition, the glass(es) of the invention may be pre-treated so that they have crystallised apatite on their surface. The surface apatite may have a crystal size less than 5 microns, preferable less than 1 micron.

Cements made from the compositions of the present invention may be used as vehicles for drug delivery. The advantages of using such cements as a vehicle for drug delivery are as follows: (i) The drug is delivered to the site at which it is intended to have its effect, for instance antibiotic drugs can be added to prevent post-surgical infections. [ii] Delivering the drug to the site of its intended effect reduces the quantity of drug that would have had to be administered if the drug were administered orally or intravenously. [iii] Given the injectability of these cements over other bone substitutes (bioglass or ceramic granules), these cements (and thus drug) can be administered less invasively. [iv] The cements set in vivo at low-temperatures and near neutral pH, this allows the incorporation of temperature and pH sensitive drugs, which is especially beneficial for delivery of peptide-based, anti-inflammatory and antibiotic drugs.

Much literature exists demonstrating drug release from calcium phosphate cements. Types of drugs that have been incorporated in calcium phosphate cements include antibiotics, anti-inflammatory, analgesic, anticancer, growth factors and other proteins. The same types of drugs may be included in the compositions of the present invention or in the liquid which is to be added to those compositions to make a cement.

Thus, the composition may comprise one or more of the following drugs: bone growth factors such as Bone Morphogenic Proteins, Bone Sialoprotein, Osteopontin, Osteonectin, Tissue Growth Factor or antibiotics such as ampicillin, amooxcillin, cephalexin, cephaloridine, sodium cephalothin, gentamicin, kanamycin and sodium penicillin.

The composition may also include various organic small molecules such as citric acid to retard and control setting as well as water soluble polymers such as poly(vinyl pyrollidone) that may be added to improve mixing and consistency and reduce easy susceptibility to water and potential washout at the implantation site.

A composition of the invention may be used to make a cement or an implant for use in dental or medical applications, for example, as a bone substitute.

Possible applications include but are not limited to: a restorative dental cement for filling teeth or the roots of teeth, for replacing alveolar bone, for injection in the treatment of osteoporotic fractures of the vertebrae including vertebroplasty, kyphoplasty, for use in spinal fusion procedures, treatment of bone cancers and bone augmentation procedures during joint replacement surgery and orthopaedic trauma cases.

A high molar mass water soluble polymer such as polyvinylpyrrolidone, polyvinyl alcohol polyethytlene oxide, polypropylene oxide or a polymer containing carboxylic acid groups such as polyacrylic acid or carboxylated cellulose may be added to the composition to improve the rheology and cohesiveness of the cement paste. Preferably, the polymer is added in a percentage by weight of 0.1 to 10%

Fillers may be added to the composition to improve radio-opacity for visualisation by X-rays. The fillers may include species based on high atomic number elements defined here as: Z>40 to include oxides, carbonates and phosphates of Sr, Ba Zn, Zr and Bi.

Setting time modifiers have been extensively added to calcium phosphate cement formulations in order to produce desirable setting times. For example, various pyrophosphate salts can be incorporated into the formulations. Pyrophosphate salts are especially useful in brushite forming CPC's to extend setting times and also inhibit the phases transition of brushite to apatite in-vivo. Hydroxylated organic acids (glycolic, pyruvic, lactic, malic, tartaric, and citric acids) and/or their sodium and calcium salts are used to modify both setting and rheological properties in CPC's. Other molecules can also be included to control setting times including orthophosphate salts (CaNaPO₄, CaKPO₄, CaHPO₄, Na₃PO₄, Na₂HPO₄, NaH₂PO₄, MgHPO₄, MgNaPO₄, MgKPO₄, K₃PO₄, K₂HPO₄, KH₂PO₄, ZnHPO₄, ZnNaPO₄, ZnKPO₄, SrHPO₄, SrNaPO₄, SrKPO₄); sulphate salts (Na₂SO₄, CaSO₄, CaSO₄.2H₂O, CaSO₄0.5H₂O, MgSO₄, K₂SO₄, ZnSO₄, SrSO₄); metal carbonates (CaCO₃, MgCO₃, Na₂CO₃, K₂CO₃, ZnCO₃, SrCO₃); metal oxides (CaO, MgO, ZnO, SrO, Na₂O, K₂O); metal halides (CaF₂, MgF₂, ZnF₂, KF, NaF, SrF₂, CaCl₂, MgCl₂, ZnCl₂, KCl, NaCl, SrCl₂); metal hydroxides (Mg(OH)₂, Ca(OH)₂, Zn(OH)₂, Sr(OH)₂, NaOH, KOH). Such modifiers may also be added to the compositions of the invention.

In a further aspect, the invention provides a cement made from a composition according to the invention.

In a further aspect, the present invention provides an implant made from a composition according to the invention.

A number of specific embodiments of the invention will now be described by way of example only with reference to the accompanying drawings of which:

FIG. 1 shows the widely accepted mechanism proposed by Hench to explain the bioactivity of bioactive glasses;

FIG. 2 shows the XRD pattern for NFRI1 glass and the resulting cement;

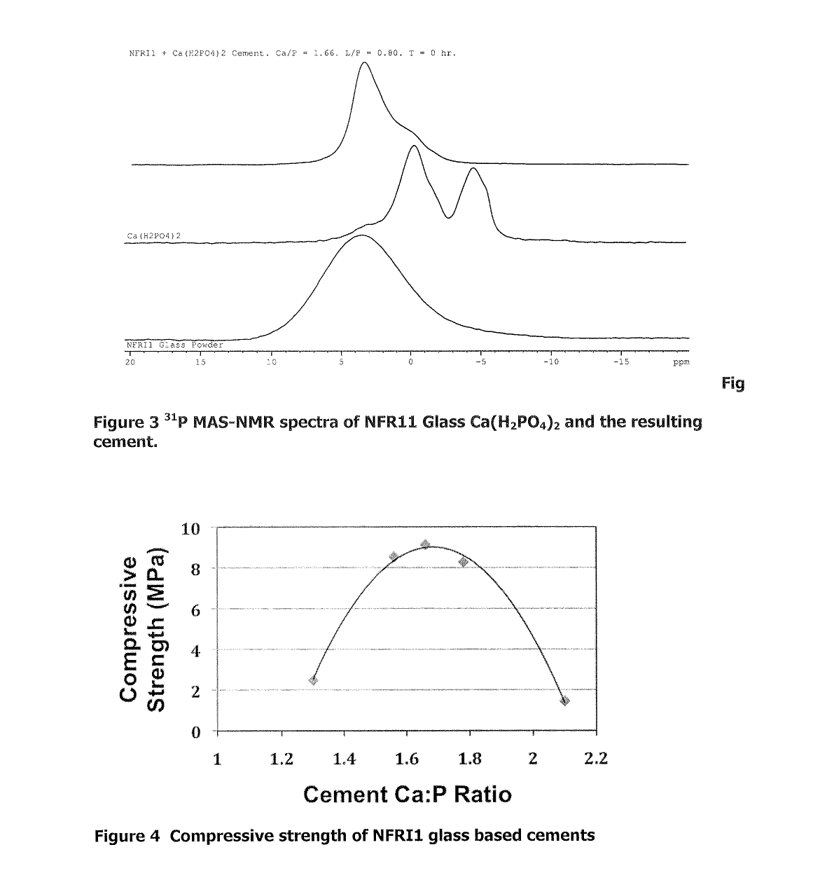

FIG. 3 shows the ³¹P MAS-NMR spectra of NFRI1 glass, Ca(H₂PO₄)₂ and the resulting cement;

FIG. 4 shows the compressive strength of NFRI1 glass based cements;

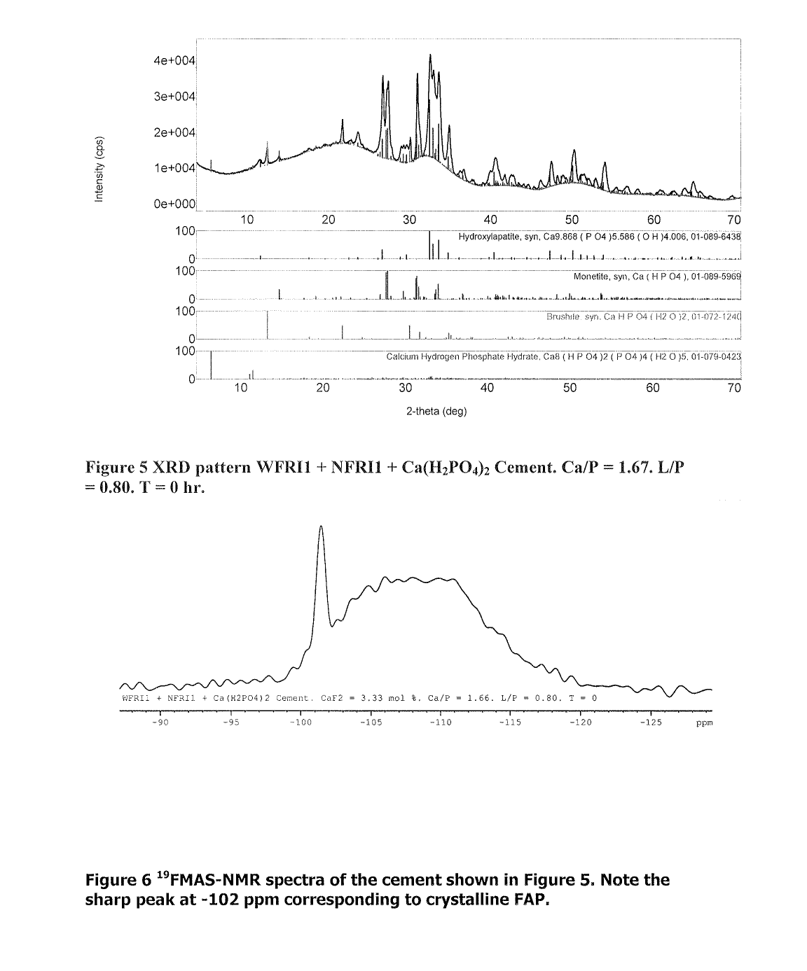

FIG. 5 shows the XRD pattern for WFRI1+Ca(H₂PO₄)₂ cement;

FIG. 6 shows the ¹⁹F MAS-NMR spectra of the cement shown in FIG. 5;

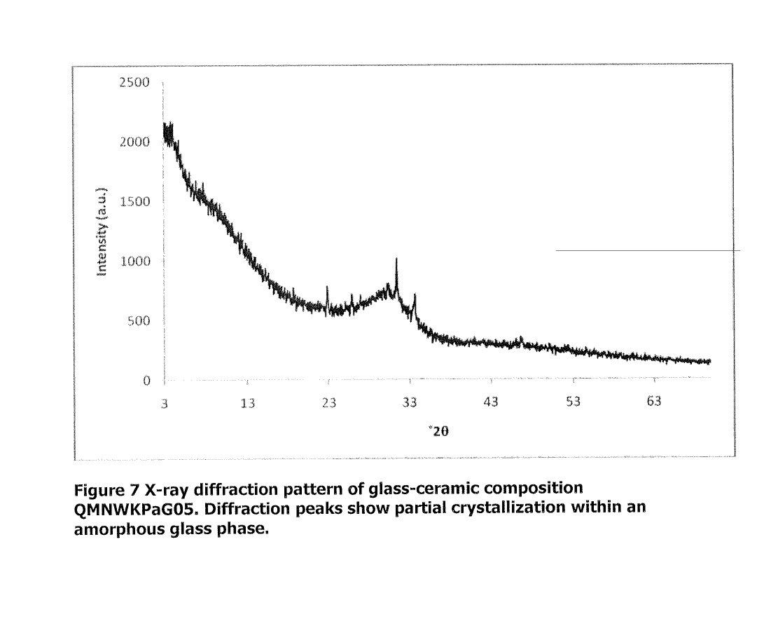

FIG. 7 shows the XRD pattern for composition QMNWKPaG05; and

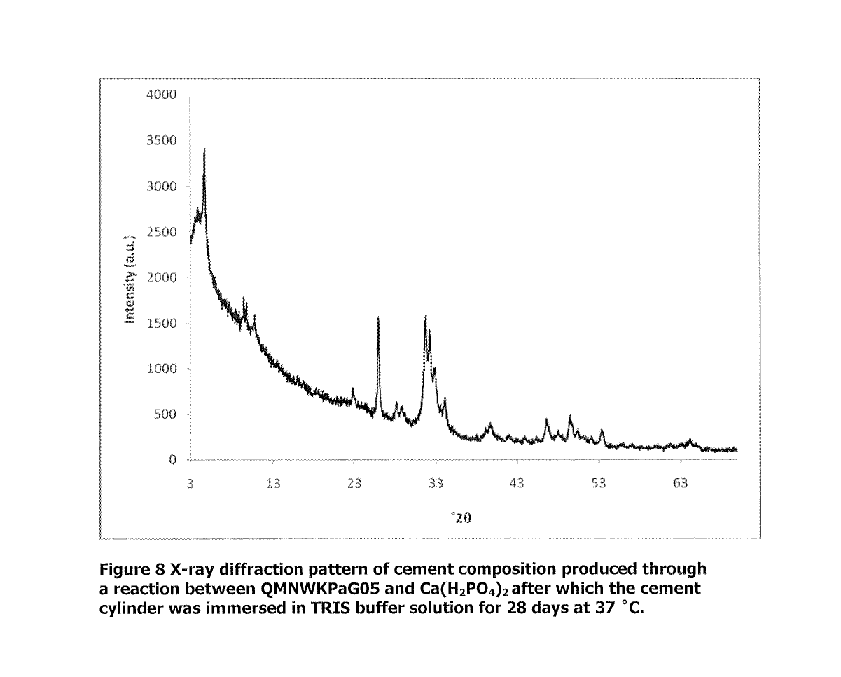

FIG. 8 shows the XRD pattern of a cement composition produced through the reaction between QMNWKPaGO5 and Ca(H₂PO₄)₂ after which the cement cylinder was immersed in TRIS buffer solution for 28 days at 37° C.

### EXAMPLES

The glass compositions shown in Tables 1a to 1d were synthesized by a melt quench route.

For each composition, appropriate amounts of the oxide and fluorides listed in Tables 1a to 1d were weighed out to give approximately 200 g of batch. In the case of the oxides of calcium, and sodium, the respective carbonates were used instead of the oxides. The batch was thoroughly mixed then placed in a 300 ml platinum/rhodium crucible. The temperature was raised to between 1350 and 1500° C. and held at that temperature for 1.5 Hrs. The resulting melt was then shock quenched into water to produced granular glass which was washed with ethanol and dried immediately at 125° C. for 1 hour. The glass was then ground in a vibratory puck mill and sieved to give a particle size less than 45 microns prior to characterization.

TABLE 1a
Silicate Glass Compositions in Mole Percent

| Glass Code | SiO₂ | P₂O₅ | CaO | Na₂O | CaF₂ | NC′ |
|---|---|---|---|---|---|---|
| NFRI1 | 36.00 | 7.00 | 52.00 | 5.00 | 0.00 | 2.00 |
| WFRI1 | 33.50 | 7.00 | 49.72 | 4.78 | 5.00 | 2.00 |
| QMNWKPaG01 | 50.00 | 0.00 | 45.00 | 5.00 | 0.00 | 2.00 |
| QMNWKPaG02 | 46.00 | 2.00 | 46.80 | 5.20 | 0.00 | 2.00 |
| QMNWKPaG03 | 42.00 | 4.00 | 48.60 | 5.40 | 0.00 | 2.00 |
| QMNWKPaG04 | 38.00 | 6.00 | 50.40 | 5.60 | 0.00 | 2.00 |
| QMNWKPaG05 | 34.00 | 8.00 | 52.20 | 5.80 | 0.00 | 2.00 |
| QMNWKPaG06 | 37.00 | 6.00 | 49.50 | 5.50 | 2.00 | 2.00 |
| QMNWKPaG07 | 36.00 | 6.00 | 48.60 | 5.40 | 4.00 | 2.00 |
| QMNWKPaG08 | 36.80 | 6.00 | 49.23 | 5.47 | 2.50 | 2.00 |
| QMNWKPaG09 | 36.50 | 6.00 | 49.05 | 5.45 | 3.00 | 2.00 |
| QMNWKPaG10 | 36.30 | 6.00 | 48.78 | 5.42 | 3.50 | 2.00 |
| QMNWKPaG11 | 35.80 | 6.00 | 48.33 | 5.37 | 4.50 | 2.00 |
| QMNWKPaG13 | 42.00 | 4.00 | 49.00 | 5.00 | 0.00 | 2.00 |
| QMNWKPaG14 | 42.00 | 4.00 | 44.00 | 10.00 | 0.00 | 2.00 |
| QMNWKPaG15 | 42.00 | 4.00 | 39.00 | 15.00 | 0.00 | 2.00 |
| QMNWKPaG16 | 42.00 | 4.00 | 34.00 | 20.00 | 0.00 | 2.00 |
| QMNWKPaG17 | 42.00 | 4.00 | 29.00 | 25.00 | 0.00 | 2.00 |

“NC” means the modified network connectivity as defined by Brauer and Hill

TABLE 1b
Phosphate Glass Compositions in Mole Percent

| Glass Code | P₂O₅ | CaO | SrO | CaF₂ | TiO₂ | Na₂0 | MgO |
|---|---|---|---|---|---|---|---|
| QMDB1 | 37.00 | 29.00 | 0.00 | 0.00 | 0.00 | 24.00 | 10.00 |
| QMDB2 | 37.00 | 28.60 | 0.00 | 0.00 | 1.00 | 23.60 | 10.01 |
| QMDB3 | 37.00 | 26.70 | 0.00 | 0.00 | 5.00 | 22.10 | 9.20 |
| QMDB4 | 37.00 | 24.40 | 0.00 | 0.00 | 10.00 | 20.20 | 8.40 |
| QMDB5 | 35.00 | 27.50 | 0.00 | 0.00 | 5.50 | 22.50 | 9.50 |
| QMRHFEI1 | 50.00 | 50.00 | 0.00 | 0.00 | 0.00 | 0.00 | 0.00 |
| QMRHFEI2 | 50.00 | 25.00 | 25.00 | 0.00 | 0.00 | 0.00 | 0.00 |
| QMRHFEI3 | 50.00 | 0.00 | 50.00 | 0.00 | 0.00 | 0.00 | 0.00 |
| TG1 | 30.00 | 35.00 | 0.00 | 10.00 | 25.00 | 0.00 | 0.00 |
| TG2 | 33.00 | 38.50 | 0.00 | 0.00 | 27.50 | 0.00 | 0.00 |

TABLE 1c
Further Phosphate Glass Compositions in Mole Percent

| Glass Code | P₂O₅ | CaO | Na₂O |
|---|---|---|---|
| QMNKPG01 | 35.00 | 43.34 | 21.67 |
| QMNKPG02 | 40.00 | 40.00 | 20.00 |
| QMNKPG03 | 45.00 | 36.66 | 18.33 |
| QMNKPG04 | 50.00 | 33.34 | 16.67 |
| QMNKPG05 | 55.00 | 30.00 | 15.00 |
| QMNKPG06 | 60.00 | 26.66 | 13.33 |
| QMNKPG07 | 65.00 | 23.34 | 11.67 |
| QMNKPG08 | 50.00 | 40.00 | 10.00 |
| QMNKPG09 | 50.00 | 30.00 | 20.00 |
| QMNKPG10 | 50.00 | 20.00 | 30.00 |
| QMNKPG11 | 50.00 | 10.00 | 40.00 |

TABLE 1d
Strontium/Cobalt-Containing Silicate Glass Compositions in Mole Percent

| | SiO₂ | P₂O₅ | CaO | Na₂O | CaF₂ | SrO | Co₂O₃ |
|---|---|---|---|---|---|---|---|
| SRGC1 | 36.00 | 7.00 | 39.00 | 5.00 | 0.00 | 13.00 | 0.00 |
| SRGC2 | 36.00 | 7.00 | 21.00 | 5.00 | 0.00 | 21.00 | 0.00 |
| SRGC3 | 36.00 | 7.00 | 13.00 | 5.00 | 0.00 | 39.00 | 0.00 |
| CCGC1 | 36.00 | 7.00 | 51.00 | 5.00 | 0.00 | 0.00 | 1.00 |

### Example 1

An octacalcium phosphate cement was prepared by mixing glass NFRI1 shown in Table 1a and Ca(H₂PO₄)₂, in a weight ratio 54:46. This powder was then mixed with 2.5% solution of Na₂HPO₄ solution to give a liquid to powder ratio of 0.8 and an overall molar ratio of Ca/:P=1.67. After the Na₂H₂PO₄ solution was pipetted into the powder mixture, the paste was mixed using a spatula on a glass slab for 30 seconds until a smooth paste was obtained. The resulting mixture was packed into split stainless steel moulds measuring 8.0 mm in diameter and 12.0 mm high. Setting time was assessed according to the ISO standard ISO (9917-1:2007(E)) using the Gilmore needle test, and is shown in Table 2. Also, strength after 1 hour in TRIS buffer solution is shown in Table 3.

TABLE 2
Setting Time (min) for NFRI1 + Ca(H₂PO₄)₂ Cement System

| | Weight Ratio | Initial Setting | Final Setting |
|---|---|---|---|
| Ca/P | (NFRI1:Ca(H₂PO₄)₂) | Time (min) | Time (min) |
| 1.30 | 40:60 | 36.00 (±0.81) | <90.00 (±0.00) |
| 1.56 | 50:50 | 9.67 (±0.94) | 26.00 (±1.15) |
| 1.67 | 54:46 | 6.00 (±1.15) | 18.67 (±1.33) |
| 1.78 | 58:42 | 31.67 (±0.88) | 37.00 (±1.00) |
| 2.10 | 66:44 | 18.67 (±0.66) | 37.00 (±1.14) |

| Weight Ratio | Initial Setting | Final Setting |
|---|---|---|
| (WFRI1:NFRI1:Ca(H₂PO₄)₂) | Time (min) | Time (min) |
| 01:49:50 | 21.0 | 30.0 |
| 03:47:50 | 23.0 | 33.0 |
| 4.5:44.5:50 | 18.5 | 31.0 |
| 8.5:41.5:50 | 19.5 | 37.0 |
| 20:30:50 | 28.0 | 39.0 |
| 50:00:50 | 28.5 | 38.5 |

TABLE 3
Strength (After 1 hr in TRIS Buffer Solution)
for NFRI1 + Ca(H₂PO₄)₂ Cement System

| | Weight Ratio | Compressive |
|---|---|---|
| Ca/P | (NFRI1:Ca(H₂PO₄)₂) | Strength (MPa) |
| 1.30 | 40:60 | 2.48 |
| 1.56 | 50:50 | 8.53 |
| 1.67 | 54:46 | 9.13 |
| 1.78 | 58:42 | 8.28 |
| 2.10 | 66:44 | 1.44 |

After setting, OCP formation was determined by performing XRD and ³¹P solid state Magic Angle Spinning Nuclear Magnetic Resonance (MAS-NMR) Spectroscopy.

FIG. 2 shows the resulting XRD pattern. The diffraction pattern is similar to that of hydroxyapatite, but has an additional sharp diffraction peak at 4.6° which corresponds to octacalcium phosphate. The presence of octacalcium phosphate was confirmed by ³¹P MAS-NMR.

FIG. 3 shows the ³¹P MAS-NMR spectra of the NFRI1 glass, Ca(H₂PO₄)₂ powder and the set cement. The glass has a broad peak at 3.7 ppm corresponding to a mixed Ca/Na orthophosphate species. The Ca(H₂PO₄)₂ exhibits two peaks at −0.4 and −4.4 ppm. The characteristic peaks for the glass and for the Ca(H₂PO₄) have disappeared in the set cement and have been replaced by peaks at 3.4 and −0.1 ppm which corresponds to octacalcium phosphate.

The compressive strength of the NFRI1 glass-based cement was measured on set cement cyclinders. Cylinders with dimensions of height: 12 mm, diameter: 8 mm were prepared and allowed to set for 2 hours in the mould. The cyclinders were then removed and placed into TRIS buffer solution for 0 hr, 1 hr, 1 d, 7 d, 14 d and 28 d prior to testing.

The compressive strength of the cylinder which has not been placed in TRIS buffer solution is shown in FIG. 4. The remaining compressive strengths are shown in the table set out below.

| Time (Hours) | Compressive Strength (MPa) |
|---|---|
| 1 | 9.13 |
| 24 | 10.07 |
| 168 | 9.90 |
| 672 | 7.66 |

NFRI1 + Ca(H₂PO₄)₂ Cement. Ca/P = 1.67. L/P = 0.80.

### Example 2

A fluorapatite cement was made in an identical fashion to Example 1 but the NFRI1 glass was replaced by the WFRI1 glass shown in Table 1a. This glass contains 5 mole % CaF₂.

FIG. 5 shows the XRD pattern of the resulting cement. The characteristic peak for octacalcium phosphate at 40 degrees two theta is now absent from the diffraction pattern and the diffraction pattern corresponds to that of fluorapatite (FAP-Ca₅(PO₄)₃F). It is impossible to distinguish between hydroxyapatite (Ca₅(PO₄)₃OH) and FAP by XRD so the presence of FAP was confirmed by ¹⁹F MAS-NMR spectroscopy. The ¹⁹F MAS-NMR spectrum for the cement is shown in FIG. 6, and shows a sharp peak at −102 ppm close to that for FAP at −101 ppm, plus a broader peak (present at −108 ppm) that indicates the presence of an amorphous fluoride-containing species that is present from the original glass.

### Example 3

A cement comprising a bioactive silicate glass and a phosphate glass was made by mixing together two powdered glasses in the ratios outlined in Table 4. The resulting cement powder was mixed with deionised water in the liquid to powder ratio (L/P) outlined in Table 4 and mixed for 30 seconds. The cement paste was then transferred to cylindrical moulds (68×4 mm), and the moulds were transferred to a 37° C. oven for 24 hours.

TABLE 4
Glass codes for cement compositions produced through reactions
between three phosphate glasses with a bioactive silicate
glass. Showing amount of each glass powder used, the L/P
ratio and whether the composition set within 24 hours.

| | | | | | Set to |
|---|---|---|---|---|---|
| | | | | Liquid to | form |
| | | Amount | Amount | Powder | cement |
| Glass Code1 | Glass Code 2 | G1 | G2 | ratio | in <24 |
| (G1) | (G2) | (g) | (g) | (ml/g) | hours |
| QMNKPG03 | QMNWKPaG04 | 0.50 | 0.50 | 0.30 | YES |
| QMNKPG04 | QMNWKPaG04 | 0.50 | 0.50 | 0.30 | YES |
| QMNKPG05 | QMNWKPaG04 | 0.50 | 0.50 | 0.35 | YES |

### Example 4

A cement comprising a bioactive silicate glass and a strontium phosphate salt was made by mixing together a powdered silicate glass and powdered SrHPO₄ in the ratios outlined in Table 5. The powdered SrHPO₄ was made by milling 1.30 g of SrHPO₄ for 4 minutes in a GyRo mill. The resulting cement powder was mixed with a 2.5% Na₂HPO₄ solution in the L/P ratio outlined in Table 5 and mixed for 30 seconds. The cement paste was then transferred to cylindrical moulds (8×4 mm), and the moulds were transferred to a 37° C. oven for 24 hours.

TABLE 5
Glass codes for cement compositions produced through
reactions between three bioactive silicate glasses with
SrHPO₄, Showing amount of each glass powder used, the
L/P ratio and whether the composition set within 24 hours.

| | | | | | Set to |
|---|---|---|---|---|---|
| | | | | Liquid to | form |
| | | Amount | Amount | Powder | cement |
| | | Salt | Glass | ratio | in <24 |
| Salt | Glass Code | (g) | (g) | (ml/g) | hours |
| SrHPO₄ | QMNWKPaG04 | 0.50 | 0.50 | 0.30 | YES |
| SrHPO₄ | QMNWKPaG08 | 0.50 | 0.50 | 0.30 | YES |
| SrHPO₄ | QMNWKPaG15 | 0.50 | 0.50 | 0.30 | YES |

### Example 5

An octacalcium phosphate cement was prepared by mixing a powdered strontium-containing bioactive silicate glass and powdered Ca(H₂PO₄)₂. The powdered Ca(H₂PO₄)₂ was made by milling 1. 30 g of Ca(H₂PO₄)₂ for 4 minutes in a GyRo mill. The glass powder and the milled Ca(H₂PO₄)₂ powder were mixed together in the ratios outlined in Table 6. The resulting cement powder was mixed with a 2.5% Na₂HPO₄ solution in the L/P ratio outlined in Table 6 and mixed for 30 seconds. The cement paste was then transferred to cylindrical moulds (8×4 mm), and the moulds were transferred to a 37° C. oven for 24 hours.

TABLE 6
Glass code for cement a composition produced through a reaction
between a strontium containing bioactive silicate glass and
Ca(H₂PO₄)₂. Showing amount of each powder used, the L/P
ratio and whether the composition set within 24 hours

| | | | | | Set to |
|---|---|---|---|---|---|
| | | | | Liquid to | form |
| | | Amount | Amount | Powder | cement |
| | | Salt | Glass | ratio | in <24 |
| Salt | Glass Code | (g) | (g) | (ml/g) | hours |
| Ca(H₂PO₄)₂ | SRGC1 | 0.50 | 0.50 | 0.70 | YES |

### Example 6

An octacalcium phosphate cement was prepared by mixing a bioactive silicate glass and powdered Ca(H₂PO₄)₂. The powdered Ca(H₂PO₄)₂ was made by milling 1. 30 g of Ca(H₂PO₄)₂ for 4 minutes in a GyRo mill. The glass powder and the milled Ca(H₂PO₄)₂ powder were mixed together in the ratios outlined in Table 7. The resulting cement powder was mixed with a 2.5% Na₂HPO₄ solution in the LIP ratio outlined in Table 7 and mixed for 30 seconds. The cement paste was then transferred to cylindrical moulds (8×4 mm), and the moulds were transferred to a 37° C. oven for 28 days.

TABLE 7
Glass code for cement a composition produced through
a reaction between a bioactive silicate glass-L/P ratio
and whether the composition set within 24 hours

| | | | | | Set to |
|---|---|---|---|---|---|
| | | | | Liquid to | form |
| | | Amount | Amount | Powder | cement |
| | | Salt | Glass | ratio | in <24 |
| Salt | Glass Code | (g) | (g) | (ml/g) | hours |
| Ca(H₂PO₄)₂ | QMNWKPaG05 | 0.53 | 0.47 | 0.70 | YES |

FIG. 7 shows the X-ray diffraction pattern for composition QMNWKPaG05 (Table 1a). The X-ray diffraction pattern shows partial crystallisation of the composition which occurred during quenching of the glass. A setting cement composition was produced from this glass composition which was placed into TRIS buffer solution. FIG. 8 shows that after 28 days immersion octacalcium phosphate was present as the cement phase.

Further examples of glasses suitable for use in compositions according to the invention are given in Tables 1a to 1d.

1. A composition for making a cement or an implant, the composition comprising a silicate glass and at least one compound selected from the group consisting of a calcium phosphate salt, a strontium phosphate salt and a phosphate glass.

2. A composition according to claim 1, wherein the silicate glass is bioactive.

3. A composition according to claim 1, wherein the silicate glass is degradable.

4. A composition according to claim 1, wherein the silicate glass contains less than 30 mole percent, preferably less than 20 mole percent, more preferably less than 10 mole percent, of an alkali metal oxide or less than 25 mole percent, preferably less than 12 mole percent, of an alkali metal fluoride.

5. A composition according to claim 4, wherein the silicate glass has a SiO₂ content below 60 mole percent.

6. A composition according to claim 1, wherein the silicate glass contains a fluoride, the fluoride content expressed as a divalent or monovalent fluoride being up to 25 mole percent, preferably up to 18 mole percent, more preferably between 0.01 and 12 mole percent, most preferably between 0.01 and 5 mole percent.

7. A composition according to claim 1, wherein the silicate glass contains a fluoride, the fluoride content expressed as a divalent or monovalent fluoride being up to 25 mole percent, preferably up to 18 mole percent, more preferably between 0.01 and 12 mole percent, most preferably between 0.01 and 5 mole percent.

8. A composition according to claim 1, wherein the silicate glass contains a metal fluoride.

9. A composition according to claim 1, wherein the silicate glass contains between 10 and 60 mole percent, preferably between 20 and 55 mole percent, more preferably between 35 and 50 mole percent, of CaO or SrO or a combination thereof.

10. A composition according to claim 1, wherein the Ca+Sr):P/molar ratio of the silicate glass lies between 0.1 and 20, preferably between 0.5 and 3.

11. A composition according to claim 1, wherein the composition comprises a source of soluble phosphate.

12. A composition according to claim 1, wherein the phosphate glass has a P₂O₅ content between 25 and 65 mole percent.

13. A composition according to claim 1, wherein the (Ca+Sr):P/molar ratio of the phosphate glass is between 0.1 and 1.5.

14. A composition according to claim 13, wherein the (Ca+Sr):P/molar ratio of the phosphate glass is between 0.4 and 0.8.

15. A composition according to claim 1, wherein the glass(es) are ground to a powder with a particle size less than 1 mm.

16. A composition according to claim 15, wherein the silicate and phosphate glasses have a particle size less than 100 microns, preferably less than 60 microns, more preferably less than 38 microns.

17. A composition according to claim 1, wherein the composition comprises a phosphate glass, and the phosphate glass and silicate glass are co-sintered together to form a glass alloy at a temperature in the range of 350-900° C.

18. A composition according to claim 1, wherein the silicate glass and/or, where present, the phosphate glass contain cobalt with a molar percentage from 0.01 to 4.0.

19. A composition according to claim 1, wherein the phosphate glass contains 10 to 60 mole percent, preferably 2 to 30 mole percent, more preferably 4 to 20 mole percent strontium.

20. A composition according to claim 1, wherein the calcium phosphate salt is calcium hydrogen phosphate.

21. (canceled)

22. (canceled)

23. (canceled)

24. (canceled)

25. (canceled)

26. (canceled)

27. (canceled)

28. (canceled)

29. (canceled)
